(19) United States
(12) Patent Application Publication
(10) Pub. No.: US 2025/0341488 A1
(43) Pub. Date: Nov. 6, 2025

(54) Systems and Methods for Battery-Less Wirelessly Powered Dielectric Sensors

(71) Applicant: The Regents of the University of California, Oakland, CA (US)

(72) Inventors: Aydin Babakhani, Santa Monica, CA (US); Yuxiang Sun, Los Angeles, CA (US)

(73) Assignee: The Regents of the University of California, Oakland, CA (US)

(21) Appl. No.: 19/199,353

(22) Filed: May 5, 2025

Related U.S. Application Data
(63) Continuation of application No. 17/287,432, filed on Apr. 21, 2021, now Pat. No. 12,320,769.
(60) Provisional application No. 62/769,166, filed on Nov. 19, 2018.
(60) Provisional application No. 62/820,770, filed on Mar. 19, 2019.

(51) Int. Cl. G01N 27/22; A61B 5/00; H02J 50/00; H02J 50/20; H02J 50/80

(52) U.S. Cl. CPC G01N 27/228; A61B 5/0031; G01N 27/221; H02J 50/001; H02J 50/20; H02J 50/80; A61B 2560/0219; H02J 2310/23

(57) ABSTRACT

Wirelessly powered dielectric sensor in accordance with embodiments of the invention are disclosed. In many embodiments, a wirelessly powered dielectric sensor includes an RF-power receiving antenna that receives electromagnetic power, a power management unit (PMU) including a capacitor to rectify and store the electromagnetic power, and a dielectric constant sensing sensor, where the PMU monitors harvested energy and operates the dielectric sensing sensor; and where the dielectric sensing sensor senses a dielectric constant of a material that is in close proximity.

### CROSS-REFERENCE TO RELATED APPLICATIONS

The current application is a continuation of U.S. patent application Ser. No. 17/287,432 entitled “Systems and Methods for Battery-Less Wirelessly Powered Dielectric Sensors”, filed Apr. 21, 2021 and published as US 2021/0356417 on Nov. 18, 2021, which is a U.S. national phase of PCT Application No. PCT/US2019/059657 entitled, “Systems and Methods for Battery-Less Wirelessly Powered Dielectric Sensors”, filed Nov. 4, 2019, which claims the benefit of and priority under 35 U.S.C. § 119 (e) to U.S. Provisional Patent Application No. 62/820,770 entitled “Systems and Methods for Battery-Less Wirelessly Powered Dielectric Sensors” filed Mar. 19, 2019, and U.S. Provisional Patent Application No. 62/769,166 entitled “Battery-Less Wirelessly Powered Dielectric Sensor” filed Nov. 19, 2018. The disclosures of U.S. patent application Ser. No. 17/287,432, PCT Application No. PCT/US2019/059657 and U.S. Provisional Patent Application Nos. 62/820,770 and 62/769,166 are hereby incorporated by reference in their entirety for all purposes.

### STATEMENT OF FEDERALLY SPONSORED RESEARCH

This invention was made with government support under Grant Number 1533688, awarded by the National Science Foundation. The government has certain rights in the invention.

### FIELD OF THE INVENTION

The present invention generally relates to wirelessly-powered dielectric sensors with on-chip antennas.

### BACKGROUND OF THE INVENTION

Over the past few decades, the number of mobile devices has increased exponentially surpassing the global population. After several generations of evolution, mobile phones now connect more than 4 billion people in the world. In recent years, significant attention is drawn to the Internet of Things (IoT). The next level connectivity extends from mobile phones or tablets to everyday objects, from household appliances to large city infrastructures. With current projections, there will be trillions of small IoT devices distributed in the environment, with sensing, computation and communication capability. To fulfil the goal of trillions of IoT devices, a low-cost IoT node is critical for the advancement of the technology. It is obvious that powering trillions of IoT devices through wire or battery is not practical. Moreover, in certain applications such as bio-implantable device, a simple task such as changing a battery my require significant undertakings including surgery and present significant potential risks and complications that may occur during the in surgery. Furthermore, it would be highly desirable to vastly reduce the size of the bio-implant by removing the need of having a bulky battery within a bio-implant.

### SUMMARY OF THE DISCLOSURE

Wirelessly powered dielectric sensors in accordance with various embodiments of the invention are disclosed. In an embodiment, a wirelessly powered dielectric sensor includes: an RF-power receiving antenna that receives electromagnetic power, a power management unit (PMU) including a capacitor to rectify and store the electromagnetic power, and a dielectric constant sensing sensor, where the PMU monitors harvested energy and operates the dielectric sensing sensor, where the dielectric sensing sensor senses a dielectric constant of a material that is in close proximity.

In a further embodiment, the PMU further included a voltage reference circuit, a comparator, a low drop-out (LDO) regulator, where the capacitor is an on-chip storage capacitor.

In still a further embodiment, the PMU monitors a voltage on the capacitor and turns a transmitter circuit on when there is sufficient energy in the capacitor.

In still a further embodiment again, the PMU generates enable signals to turn on the low drop-out regulator to generate a regulated voltage V_{reg} for the dielectric sensing sensor and to turn on the dielectric sensing sensor.

In another additional embodiment, the receiving antenna is an on-chip antenna. In still another embodiment again, the capacitor is an on-chip capacitor.

In another additional embodiment, the wirelessly powered dielectric sensor further includes a transmitting on-chip antenna, where the transmitting on-chip antenna is used to wirelessly transmit a signal.

In still a further embodiment, the dielectric sensing oscillator drives the transmitting on-chip antenna to radiate back a signal.

In still a further embodiment again, the transmitting on-chip antenna is used to transmit the signal using at least one of a wired communication channel or a wireless communication channel.

In still a further embodiment, the dielectric constant sensing sensor is an oscillator that produces a frequency shift depending on the value of the dielectric constant being measured.

In still a further embodiment again, the PMU operates the dielectric sensing sensor in duty cycle mode.

In still a further embodiment again, the dielectric sensing sensor is used to receive a command where there is a nonconductive isolating layer between a user providing the command and the wirelessly powered dielectric sensor.

In still another additional embodiment, the dielectric sensing sensor includes a metaloxide-metal capacitor (MOMCAP) that provides different capacitance for different materials.

In another embodiment includes a method for wirelessly powering a dielectric sensor, including receiving electromagnetic power using an RF-power receiving antenna, rectifying and storing the electromagnetic power using a capacitor included in a power management unit (PMU), sensing a dielectric constant using a dielectric constant sensing sensor, where the PMU monitors harvested energy and operates the dielectric sensing sensor, where the dielectric sensing sensor senses a dielectric constant of a material that is in close proximity.

In a further embodiment, the PMU further includes a voltage reference circuit, a comparator, a low drop-out (LDO) regulator, wherein the capacitor is an on-chip storage capacitor.

In still a further embodiment, the PMU monitors a voltage on the capacitor and turns a transmitter circuit on when there is sufficient energy in the capacitor.

In still a further embodiment, the PMU generates enable signals to turn on the low drop-out regulator to generate a regulated voltage V_{reg} for the dielectric sensing sensor and to turn on the dielectric sensing sensor.

In still a further embodiment again, the receiving antenna is an on-chip antenna.

In still a further embodiment again, the capacitor is an on-chip capacitor.

In still another embodiment again, the dielectric sensor includes a transmitting on-chip antenna, where the transmitting on-chip antenna is used to wirelessly transmit a signal.

### BRIEF DESCRIPTION OF THE DRAWINGS

FIG. 1 illustrates self-interference in an RFID system in accordance with several embodiments of the invention.

FIG. 2 illustrates a wirelessly powered IoT sensor system with frequency division scheme to solve self-interference in accordance with several embodiments of the invention.

FIG. 3 illustrates a block diagram of a wirelessly-powered dielectric sensor chip in accordance with several embodiments of the invention.

FIG. 4 illustrates a transient diagram of voltages in duty-cycled operation in accordance with several embodiments of the invention.

FIG. 5 illustrates wirelessly powered harvesting scheme, with TX antenna and power receiving front-end: RX antenna, matching circuit, and rectifier in accordance with several embodiments of the invention.

FIG. 6 illustrates on-chip dipole antenna structure EM simulation in HyperLynx 3D EM in accordance with several embodiments of the invention.

FIG. 7 illustrates on-chip dipole antenna radiation efficiency versus frequency in accordance with several embodiments of the invention.

FIG. 8 illustrates radiation pattern of on-chip RX antenna directivity at 10 GHz in accordance with several embodiments of the invention.

FIG. 9 illustrates on-chip dipole antenna impedance versus frequency in accordance with several embodiments of the invention.

FIG. 10 illustrates antenna quality factor and matching circuit efficiency versus frequency in accordance with several embodiments of the invention.

FIG. 11 illustrates overall DC output power at rectifier output versus frequency in accordance with several embodiments of the invention.

FIG. 12 illustrates rectifier conversion efficiency versus rectifier input power in accordance with several embodiments of the invention.

FIG. 13a illustrates schematics of voltage reference and voltage divider in accordance with several embodiments of the invention.

FIG. 13b illustrates schematics of comparator in accordance with several embodiments of the invention.

FIG. 13c illustrates schematics of LDO in accordance with several embodiments of the invention.

FIG. 14a illustrates MOMCAP structure in 180 nm CMOS top metal layer in accordance with several embodiments of the invention.

FIG. 14b illustrates capacitance with oil and epoxy on top, versus frequency in accordance with several embodiments of the invention.

FIG. 14c illustrates capacitance and oscillation frequency versus dielectric constant of material on top in accordance with several embodiments of the invention.

FIG. 15 illustrates a schematic of dielectric sensing VCO with TX on-chip antenna in accordance with several embodiments of the invention.

FIG. 16 illustrates measurement setup with oil on top of the chip in accordance with several embodiments of the invention.

FIG. 17 illustrates measured transient signal of voltage on storage (Vdd) and regulated voltage after LDO (Vreg) in accordance with several embodiments of the invention.

FIG. 18 illustrates transmitted signal spectrum from wirelessly-powered dielectric sensor, showing oscillation frequency of 4 GHZ, 3.75 GHZ, 3.66 GHz, with air, oil and epoxy on top of the chip respectively in accordance with several embodiments of the invention.

FIG. 19 illustrates a micrograph of the 3.9×0.7 mm² chip in accordance with several embodiments of the invention.

### DETAILED DESCRIPTION OF THE DRAWINGS

The internet of things (IoT) has progressed rapidly and providing for the ability to both wirelessly power and communicate with these devices has become essential in furthering the advancement of this technology. Accordingly, many embodiments provide for a wirelessly-powered dielectric sensor. In particular, many embodiments provide a wirelessly powered dielectric sensor microchip fabricated in 180 nm CMOS process for material detection and monitoring. In many embodiments, the dielectric sensor chip includes a receiving and a transmitting antenna, a RF-DC rectifier, a dielectric constant sensing sensor and a power management unit (PMU) that includes a voltage reference circuit, a comparator, a low drop-out regulator (LDO) and on-chip storage capacitor. In many embodiments, the dielectric sensing sensor oscillates at different frequencies depending on a kind of material on top of the sensing capacitor. In several embodiments, the dielectric constant detection is achieved by sensing capacitance change of a capacitor in an oscillator, which causes a shift in the oscillation frequency. In several embodiments, to power the chip, the dielectric sensor chip harvests electromagnetic energy from a continuous-wave source using an on-chip antenna, thus reducing the size of the whole sensor to a millimeter scale. In many embodiments, the dielectric sensor chip radiates back the signal to an external reader antenna. In many embodiments, the dielectric sensor chip may use a frequency division architecture that resolves the conventional self-interference issue in radio frequency ID (RFID) sensors by separating the received and transmitted frequencies.

A dielectric sensor chip in accordance with many embodiments may include applications in 3D gesture sensing for mobile devices, blood sensing in human body implant, oil and gas leakage sensing, hazardous gas sensing among various other fields as appropriate to the requirements of specific applications in accordance with embodiments of the invention. A dielectric sensor chip in accordance with many embodiments may also include applications in medical implants for leak detection, bleeding detection, tumor detection, wound healing, among various other applications.

A dielectric sensor chip in accordance with many embodiments may also include applications in consumer electronics such as smart phones displays and computer displays providing the ability to sense 3D gestures through a touchless interactions of the user with the display, whereby the smart phone is able to sense gestures without a user actually touching the screen or display of the smart phone or other electronic device. In particular, a dielectric sensing may be used to receive commands or users finger gesture where there is a nonconductive isolating layer between the user's finger and the display, for example, a user wearing a glove will still be able to provide commands to the touchscreen of their smart phone. Likewise, a user may not need to actually touch the display of their electronic device with their finger but can have the finger hover above the display to interact with the user interface. Within a 3D gesture sensing context, when a target such as finger or hand presents above a microchip in accordance with various embodiments, the effective dielectric constant of the sensing MOMCAP may be changed, hence the frequency shift can be sensed. The miniaturized sensor chip in accordance with several embodiments can also be utilized in a large array to form a dielectric constant map, which can handle more complex gesture recognition.

A dielectric sensor chip in accordance with many embodiments may also be used in applications within extreme environments where it would be difficult to implement the sensor with wires. For example, a dielectric sensor may be used inside of a high pressure oil or gas pipeline to sense and measure flow properties and send this information to an external receiver. Other applications include, for example, using in oil and gas reservoirs, using within cement to sense whether the cement has cured or not, among various other applications that would benefit from providing sensing capabilities in extreme environments.

In particular, in environment monitoring applications, a wirelessly-powered dielectric sensor in accordance with several embodiments can be used to detect oil or gas leakage along a pipe, measure any one of a variety of variables, including flow, temperature, volume, among various other measurements appropriate to the particular applications, as well as hazardous gas sensing in a lab environment.

As noted above, dielectric sensor chip in accordance with various embodiments can be applied to a human body implant for real-time monitoring such as detecting when a patient is bleeding or detecting a leak in an implant.

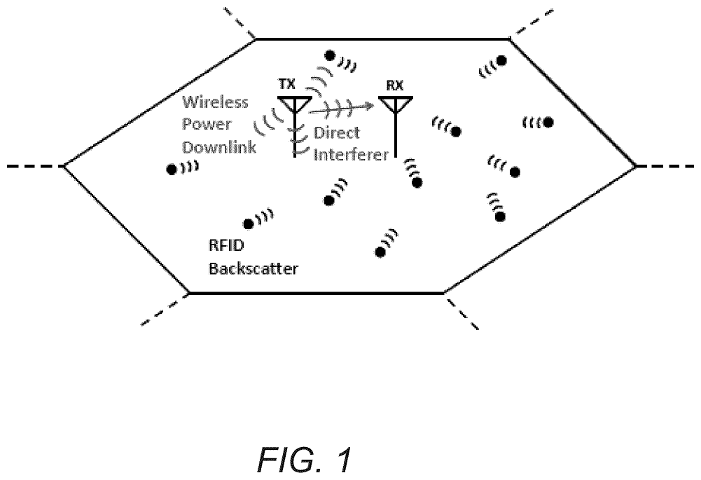

A microchip in accordance with various embodiments can be wirelessly powered by RF power from a transmitting antenna. The miniaturized battery-less microchip in accordance with various embodiments can be applied in large amount distributed in the environment. The microchip may detect a target material by measurement of dielectric constant change in the near field (e.g., ˜2 cm). As illustrated in FIG. 1, a microchip may include an RF-power receiving on-chip antenna, a rectifier, a power management unit, a dielectric sensing sensor and a transmitting antenna. In certain embodiments, the RF power receiving front-end can be optimized at 9.8 GHZ, which can receive continuous rectified DC power of ˜100 μW, with a distance of ˜4 cm. In several embodiments, the power management unit may monitor the harvested energy and operate the dielectric sensing sensor in duty cycle. In several embodiments, the oscillator may sense the dielectric constant change by a customized 200 um×200 um metal-oxide-metal capacitor (MOMCAP). In certain embodiments, the oscillation free-running frequency may be 4 GHZ, and may transmit the signal back to external receiver. The frequency shifts can correspond to kinds of material with different dielectric constant. Many embodiments may map a measured dielectric constant back to possible material in different applications. In certain embodiments, the sensing distance can be 0 to ˜2 cm away from the chip. In other embodiments, the sensing distance can be greater than 2 cm away from the chip as appropriate to the requirements of specific applications in accordance with embodiments of the invention.

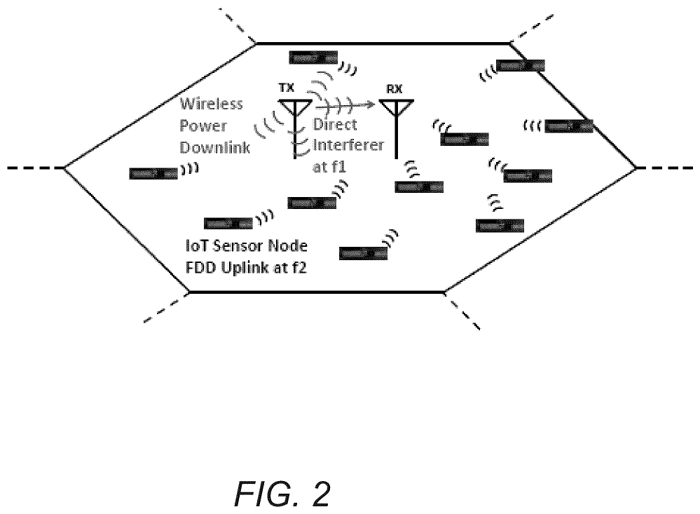

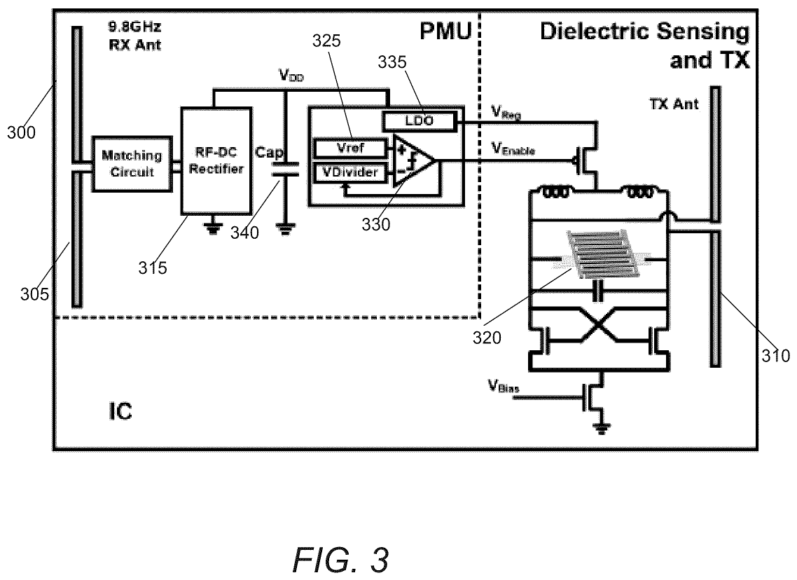

A configuration of wirelessly powered microchip operation in accordance with several embodiments is illustrated in FIG. 2. In many embodiments, the power source may transmit RF power to the microchip sensors. Each microchip can transmit back the signal to a reader. By detecting if the oscillation frequency shifts or not, many embodiments can localize and map the targeted object. A preliminary microchip pixel can be fabricated in 180 nm CMOS process as illustrated in FIG. 3 in accordance with several embodiments of the invention. The microchip can be wirelessly powered, and may send back a signal at a different frequency from 3 GHz to 4 GHZ, when there are different materials placed on the top of the chip. The miniaturized microchip may be only 3.9×0.7 mm² in size.

Accordingly, many embodiments of the invention provide a battery-less mm-sized wirelessly-powered dielectric sensor with on-chip antennas in 180 nm SOI CMOS process. The dielectric constant detection can be achieved by sensing capacitance change of a capacitor in an oscillator, which may cause a shift in the oscillation frequency. In certain embodiments, the chip may harvest electromagnetic energy from a continuous-wave source at 9.8 GHz using an on-chip antenna, which may shrink the whole sensor size to millimeter scale. In several embodiments, the oscillator free-running frequency can be from 3.66 GHz to 4 GHz which may depend on the material on top of the chip. The chip may radiate back the signal to an external reader antenna.

There are several approaches to wirelessly-power a device, such as far-field electromagnetic radiation or near-field inductive coupling, ultrasonic power, thermalelectricity, photovoltaic (PV) or optical power, among various others. Far-field electromagnetic power transfer may be a technique for IoT devices application due to its high power transmission and potential high data rate capacity. Moreover, far-field wireless power transferring at higher frequency in GHz range may allow small antenna size and large range to node size ratio, which greatly benefits IoT device miniaturization. For commercial near-field inductive coupling, it usually may need large external receiving coils, which mainly limits the miniaturization of the sensor node. The operating distance may also be restricted in order to have higher coupling coefficient. Similar miniaturization challenges may also be presented to Radio Frequency Identification (RFID) systems. Conventional RFID sensors typically operate in the sub-gigahertz frequency regime and therefore may require large external antennas with an area exceeding 10 cm\hat{ }2. This may severely limit the miniaturization of the device and cause complex packaging issues and increased cost. Another challenge is that RFID may apply a backscatter modulation scheme to transmit back signal, leading to a serious self-interference issue. The large power transferring downlink may act as a blocker for uplink backscatter signal. FIG. 1 illustrates receiving a weak reflected signal with an RX antenna, where there can be interference at the same frequency that is coupled from TX to RX, which may greatly degrade the sensitivity and signal-to-noise ratio (SNR) of a reader's receiver. Accordingly, many embodiments of the dielectric sensor chip mitigate the self-interference issue using frequency division to separate downlink and uplink frequency and time division duplexing.

Accordingly, many embodiments provide a wirelessly-powered frequency shift based dielectric sensor. In certain embodiments, the dielectric sensor may harvest power at 9.8 GHz. In order to shrink the size of the entire sensor node, many embodiments integrate the antenna with the energy-harvesting circuits, which may dramatically reduce the overall system size to the millimeter level. In numerous embodiments, the dielectric constant change can be sensed by a customized metal-oxide-metal capacitor (MOMCAP) in the oscillator. The oscillation frequency may change from 3.66 GHz to 4 GHz in the measurement when different materials are placed on the top of the chip. The oscillator may drive another TX on-chip antenna to transmit the signal back. The rectifier front-end and power management unit may also be integrated in accordance with several embodiments of the invention. The whole chip may occupy an area of 2.73 mm² including on-chip antennas.

Described below are architectures of circuits for IoT sensor applications in accordance with many embodiments of the invention. Furthermore, details of circuit designs and optimizations of wireless energy harvesting on-chip antennas and rectifiers, power management units and dielectric sensing sensors in accordance with numerous embodiments are discussed. In many embodiments, the circuit was taped out in a 180 nm SOI CMOS process and tested.

### System Architecture

In many embodiments, in order to minimize the self-interference issue, a frequency division duplexing scheme may be adopted. FIG. 2 illustrates a wirelessly powered IoT sensor system with frequency division scheme to solve self-interference issue in accordance with several embodiments of the invention.

As illustrated in FIG. 2, the IoT sensor node's transmitted frequency can be set at frequency f2, which may be different than the signal received at frequency f1 from the base station transmitter. This frequency division may eliminate the self-interference and increases the dynamic range of the receiver at the reader. Although FIG. 2 illustrates a particular wirelessly powered IoT sensor system with a frequency division scheme, any of a variety of frequency division schemes may be utilized as appropriate to the requirements of specific applications in accordance with embodiments of the invention.

A dielectric sensor chip may include circuitry for both wirelessly powering the chip and circuitry for sensing and transmitting a signal to a receiver antenna. FIG. 3 illustrates a block diagram of a wirelessly-powered dielectric sensor node in accordance with several embodiments of the invention. As illustrated, the dielectric sensor chip 300 may include a receiving on-chip dipole antenna 305 and a transmitting on-chip dipole antenna 310, a RF-DC rectifier 315, a frequency-shift dielectric constant sensing sensor 320 and a power management unit (PMU) which can include a voltage reference circuit 325, a comparator 330, a low drop-out regulator (LDO) 335, an on-chip storage capacitor 340.

The on-chip antenna may receive the incoming electromagnetic power and may feed it to the matching circuit and energy harvesting rectifier. The power may be rectified and stored in an on-chip capacitor. The PMU unit may continuously monitor the voltage on the storage capacitor and may turn the transmitter circuit on after the chip scavenges and stores sufficient energy in the storage capacitor. In several embodiments, when the voltage on the storage capacitor (V_{DD}) reaches a particular threshold, such as for example 1.6 V (V_{High}) in certain embodiments, the PMU may generate enable signals (V̅_̅{̅E̅n̅a̅b̅l̅e̅}̅), to turn on the low drop-out regulator to generate a regulated voltage (V_{Reg}) for oscillator. The PMU also may turn on the dielectric sensing oscillator, which may oscillate at frequency from a particular range, for example 3.66 GHz to 4 GHz in certain embodiments, depending on the kind of material on top of the sensing capacitor. In many embodiments, the oscillator may drive the TX on-chip antenna and radiate back the signal. This event may discharge the storage capacitor. In several embodiments, when the capacitor voltage drops to a particular threshold, for example 1.2 V (V_{Low}), the PMU turns the oscillator off and the chip enters the sleep mode. Although FIG. 3 illustrates a particular circuit architecture of a wirelessly-powered dielectric sensor chip, any of a variety of circuit architectures may be utilized as appropriate to the requirements of specific applications in accordance with embodiments of the invention.

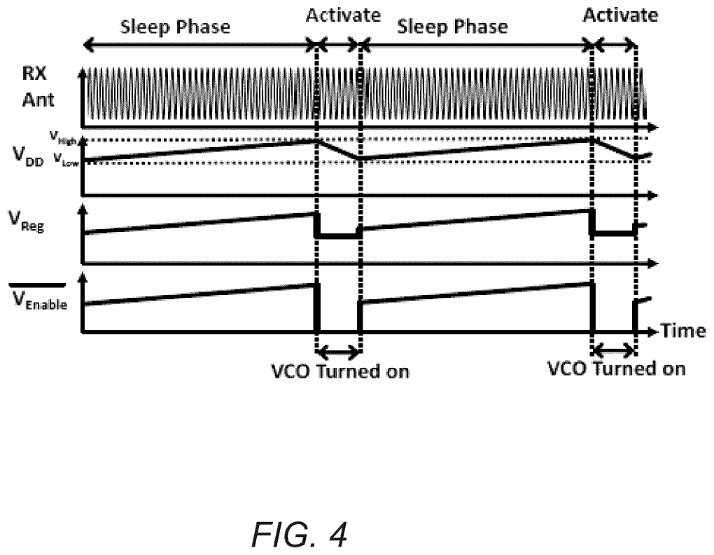

FIG. 4 illustrates a timing diagram of a duty-cycled operation in accordance with various embodiments of the invention. Applying duty-cycled operation may allow higher power consumption of the oscillator, while low wireless power can be harvested.

### Circuit Design and Optimization of Rf Powered Dielectric Sensor

Discussed below are RX on-chip antenna designs that may be utilized within dielectric sensor chips together with rectifiers in accordance with several embodiments of the invention. Many embodiments may choose the power down-link frequency to achieve an optimum total power conversion efficiency, where maximum DC power is rectified at output storage capacitor when fixing the RF source power and the distance between the source and the receiver front-end. Then, the power management circuit may be designed to operate the sensor node in duty cycle mode in accordance with numerous embodiments. Finally, a dielectric sensing capacitor and oscillator may be simulated and analyzed.

### RF Rectifier Antenna Co-Design and Frequency Optimization

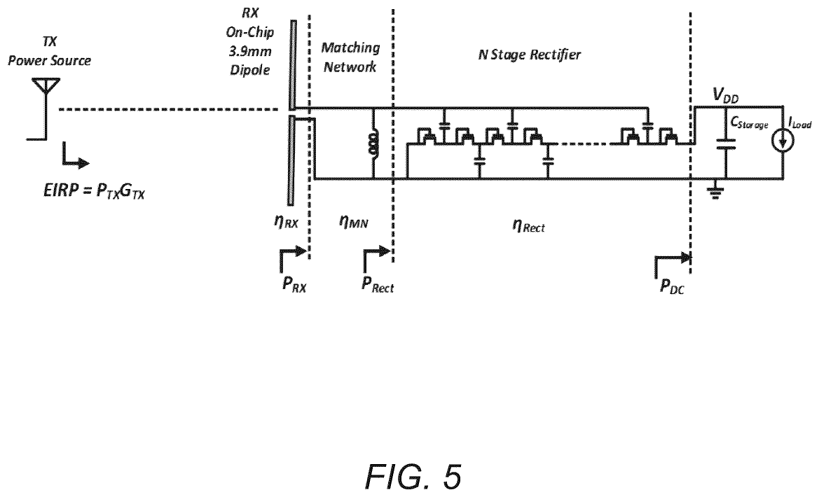

Many embodiments provide for the wireless powering of the dielectric sensors. FIG. 5 illustrates a wirelessly powered harvesting scheme, with TX antenna and power receiving front-end: RX antenna, matching circuit, and rectifier in accordance with several embodiments of the invention.

For a common RF power front-end structure as shown in FIG. 5, a maximum converted DC power at the rectifier output may be desired. The converted DC power (P_{DC}) may be a function of the received power from the antenna (P_{RX}), matching network circuit efficiency (η_{MN}), and rectifier conversion efficiency (η_{Rect}).

$${P}_{DC} = {P}_{RX} {η}_{m} \text{?} \qquad ( 1 )$$

$$\text{?} \text{indicates text missing or illegible when filed}$$

The receiver antenna's received power can be depicted by the Friis equation:

$${P}_{RX} = \frac{{P}_{TX} {G}_{TX}}{4 π {d}^{2}} \frac{{G}_{RX} {λ}^{2}}{4 π} = \frac{{EIRP}_{TX}}{4 π {d}^{2}} \frac{{η}_{RX} {D}_{RX} {λ}^{2}}{4 π} \qquad ( 2 )$$

where P_{T}, G_{TX}, EIRP_{TX} are transmitting power, gain and equivalent isotropically radiated power of the transmitting antenna respectively, λ is the wavelength, G_{RX} is the gain of the receiving antenna, d is the distance between TX and RX, DRX is the directivity of the receiver antenna. ηRX is the radiation efficiency of receiving antenna. The matching circuit efficiency may be defined as:

$${η}_{MN} = \frac{{P}_{Rect}}{{P}_{RX}} \qquad ( 3 )$$

where P_{Rect} is the power delivered to the rectifier input. While the rectifier conversion efficiency can be defined as:

$${η}_{Rect} = \frac{{P}_{DC}}{{P}_{Rect}} \qquad ( 4 )$$

Many embodiments investigate into each of the three stages and optimize the operating frequency in order to get the most rectified DC power to the storage capacitor.

### Receiver Antenna

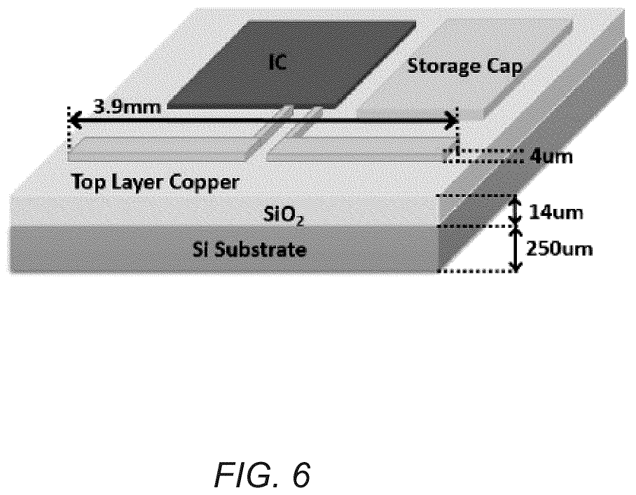

In order to miniaturize the size of a sensor node, many embodiments of the dielectric sensor chip may integrate one or more on-chip antennas. Due to the limited die dimension, in many embodiments, the on-chip receiving antenna may be implemented as a dipole antenna. In certain embodiments, the dipole antenna may have a length of 3.9 mm. The received power may suffer from the trade-off of path loss and radiation efficiency. As frequency becomes higher and closer to its resonant frequency, the power link may have more path loss but benefit from a greater radiation efficiency of the receiving antenna. In several embodiments, the antenna may be implemented on a 250-m thick silicon substrate and a 14-m thick silicon dioxide layer. FIG. 6 illustrates an antenna implemented on a 250-m thick silicon substrate and a 14-m thick silicone dioxide layer in accordance with a particular embodiment of the invention. An EM simulation of this structure by Hyperlynx 3D EM was done to characterize the on-chip dipole antenna. Although FIG. 6 illustrates a particular di-pole antenna design, any of a variety of antenna designs at different sizes may be implemented as appropriate to the requirements of specific applications in accordance with embodiments of the invention.

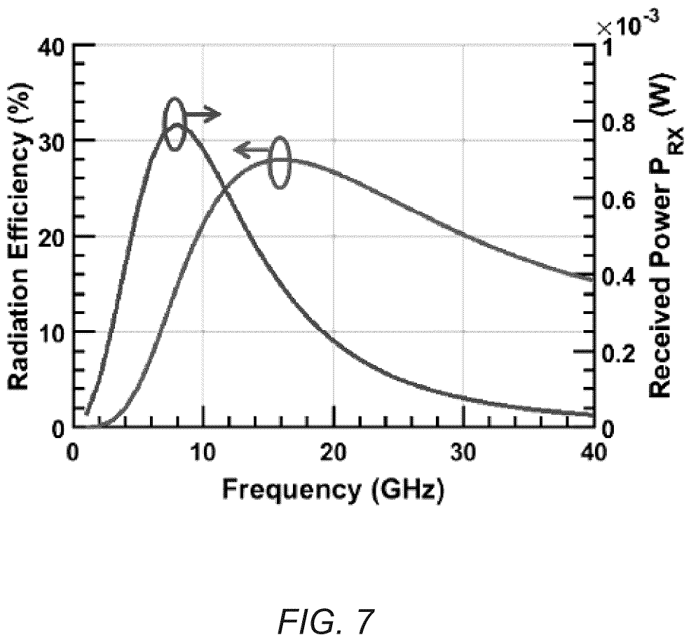

FIG. 7 illustrates a simulated radiation efficiency in accordance with an embodiment of the invention. In certain embodiments, the dipole antenna may achieve a maximum radiation efficiency of 28% at 17 GHZ. In 180 nm SOI CMOS process, the silicon substrate can have a conductivity of only 0.1 S/m. Due to this low substrate loss, the radiation efficiency is not degraded too much.

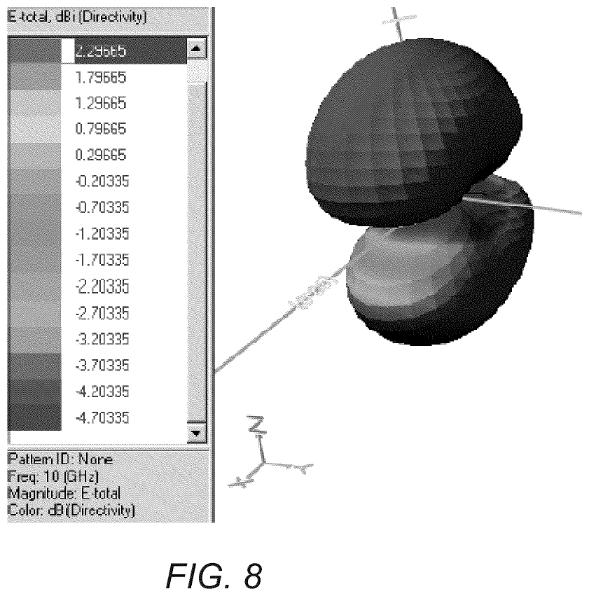

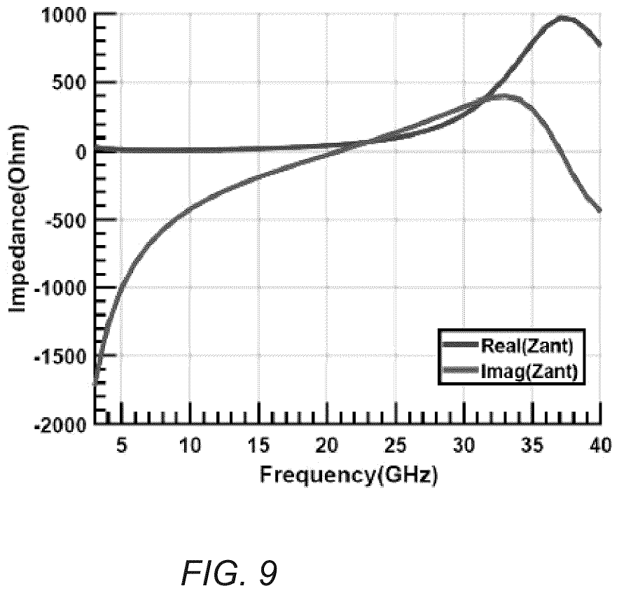

In the case where EIRP of the transmitter antenna is 1 W and the distance between the TX and the sensor node is 5 cm, the received power is shown in FIG. 7. After taking into account of the path loss, the maximum point can be shifted to around 8 GHz. A 3D radiation pattern of the antenna directivity at 10 GHz in accordance with several embodiments is illustrated in FIG. 8, with a maximum directivity of 2.2 dBi. The dipole antenna impedance in accordance with several embodiments is shown in FIG. 9. The quality factor of the antenna may play an important role in matching efficiency which is described below. Further details for designing RF wireless power receiving on-chip antennas as related to FIGS. 3, 5, 6, and 14 can be found in Pozar, David M. “Microwave engineering” John Wiley & Sons, 2009, Chapter 14, “Introduction to Microwave Systems”, starting on page 658, and in Balanis, Constantine A. “Antenna theory: analysis and design”, John Wiley & sons, 2016, Chapter 2, “Fundamental Parameters of Antennas” starting on page 27 and Chapter 4 “Linear Wire Antennas” starting on page 151, and in Rahmat-Samii, Yahya, and Jaehoon Kim. “Implanted antennas in medical wireless communications.” Synthesis Lectures on Antennas 1.1 (2005): 1-82. The disclosures of which are hereby incorporated by reference herein in their entirety.

### Matching Circuit

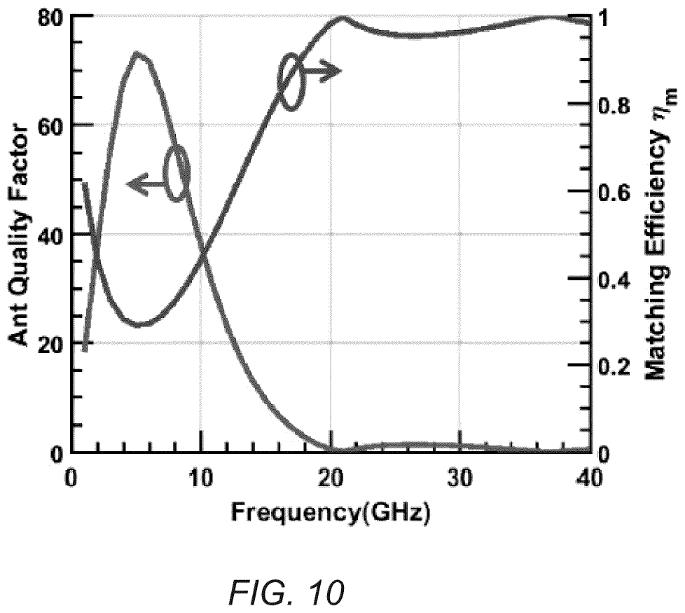

In many embodiments of the dielectric sensor, a single L matching topology may be used, due to its simplicity. Also, more complex matching topologies such as IT-matching or T-matching, with more lossy on-chip passive components may cause lower efficiency. Due to the lossy component in matching network, matching efficiency can usually not be maximized at conjugate match between RX antenna and rectifier. Based on equation 5 and 6 below, the matching circuit efficiency (ηₘ) can be a function of the quality factor of the matching component and the quality factor of the receiver antenna. In simulation, certain embodiments fix the matching component quality factor as 15, and based on the simulated on-chip dipole antenna impedance and quality factor, the matching efficiency is plotted in FIG. 10 in accordance with several embodiments. In certain embodiments, a frequency around dipole's half wavelength resonance frequency 21 GHz is preferable, where the receiver antenna quality factor is small.

$${η}_{m} = \frac{{Q}_{C}^{2} + 1}{{Q}_{C}^{2} + {Q}_{C} \text{?}} , when \text{?} > \frac{2 {Q}_{C}}{{Q}_{C}^{2} - 1} \qquad ( 5 )$$

$${η}_{m} = \frac{2}{1 + \sqrt{1 + \text{?}}} , when \text{?} < \frac{2 {Q}_{C}}{{Q}_{C}^{2} - 1} \qquad ( 6 )$$

$$\text{?} \text{indicates text missing or illegible when filed}$$

### Rectifier:

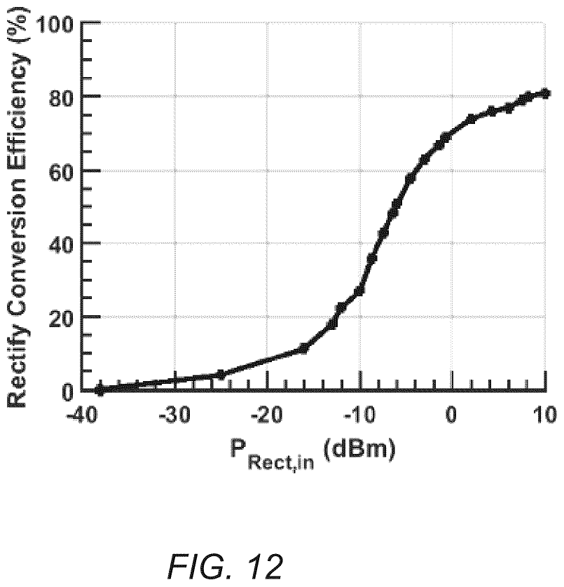

Many embodiments of the dielectric sensor chip may include a rectifier. The rectifier in accordance with several embodiments may be implemented as multiple-stage Dickson rectifier. The conversion efficiency of the rectifier may be a weak function of frequency in the operating frequency band. However, conversion efficiency can be a strong function of input power. The conversion efficiency of a ten-stage Dickson rectifier versus input power at 10 GHz is simulated and plotted in FIG. 12 in accordance with several embodiments of the invention.

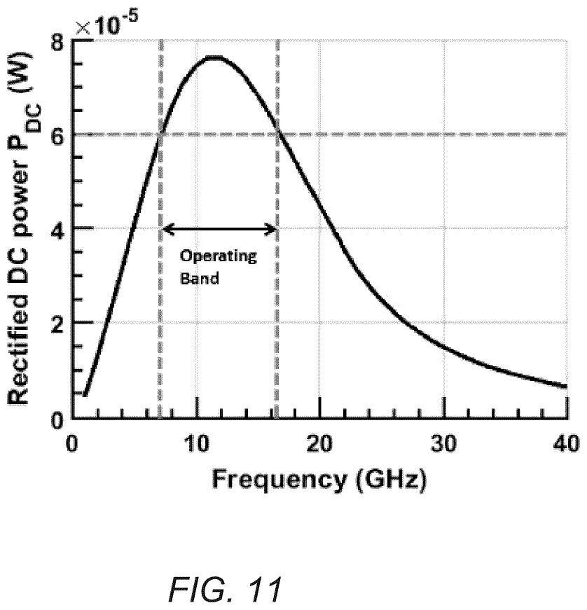

Considering all three of these stages, the overall DC output power at the rectifier output versus frequency is shown in FIG. 11 in accordance with an embodiment of the invention. As a trade off a path loss, RX radiation efficiency and matching efficiency, the optimum operating frequency band may be around 10 GHz.

Based on the optimum operating frequency, a ten-stage Dickson rectifier can be implemented. A 3.4 nH inductor may be used at the input to resonate with the dipole antenna and maximize the amplitude of the input voltage. In several embodiments, to achieve a DC voltage of 1 V on a 1.1 nF MIM storage capacitor, a minimum received power of −16.1 dBm may be used. At this input power level, a conversion efficiency of 7% at 10 GHz can be achieved.

### Power Management Unit

In several embodiments of the dielectric sensor, because there may be a gap between the μW level of the harvested power and the mW level of the oscillator's power consumption, a power management unit (PMU) may be needed to monitor the supply voltage and operate the oscillator in duty cycles.

In many embodiments, the PMU can be composed of a voltage reference, a comparator, a divider, and a low dropout regulator (LDO). In several embodiments, first, in the sleep mode, the PMU turns off the oscillator. The voltage reference circuit may generate a reference voltage, for example V_{ref}, of 0.25 V, while the voltage divider chain generates the voltage, V_{div}, which may be a ratio of the V_{DD}. Slowly, the storage capacitor can be charged. When V_{DD} reaches a high threshold, for example of 1.6 V, and the V_{div} becomes greater than V_{ref}, the comparator may generate an active output enable signal (V̅_̅{̅E̅n̅a̅b̅l̅e̅}̅). The enable signal may positively feed back to the voltage divider, leading to a larger dividing ratio and V_{div}, which further switches on the enable signal. The enable signals may turn on the oscillator and low-dropout regulator that generates a particular, e.g. 1 V, regulated supply voltage. Subsequently in the work mode, the oscillator may quickly discharge storage capacitor and V_{DD} drops. When V_{DD} reaches to a lower threshold, e.g. of 1.2 V, and V_{div} becomes smaller than V_{ref}, the enable signals may be switched off. The oscillator and LDO may revert back to sleep mode.

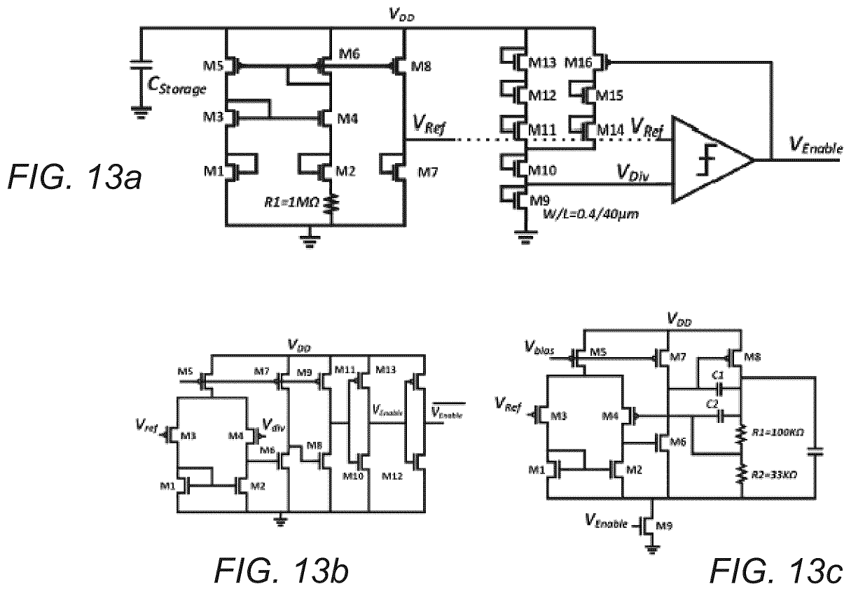

In many embodiments, the voltage reference may operate in a subthreshold region. Based on the relation that current following through M1 M2 transistor may have an exponential relation with its V_{gs}, the voltage on resistor R1 may be only a function of transistor size ratio, which can be independent of V_{DD} voltage. In a voltage divider, the diode connected transistor can be set to have a small W/L ratio to gain a large equivalent resistance, thus reducing the power consumption. In several embodiments, the LDO may be implemented with a two stage error amplifier. Compensation can be achieved with capacitor C1 and C2. In certain embodiments, the LDO's power consumption can be 22 μW, but it may be only awake when the oscillator is turned on. In certain embodiments, its leakage current can be only 300 pA when in sleep mode, which is negligible to the power consumption of other PMU circuits. The schematics of each block are shown in FIGS. 13a, 13b, and 13c in accordance with several embodiments of the invention. In particular, FIG. 13a illustrates a schematics of a voltage reference and voltage divider, FIG. 13b illustrates a schematics of a comparator and FIG. 13c illustrates schematics of an LDO. Although FIGS. 13a, 13b, and 13c illustrates a particular schematics for a voltage reference and voltage divider, a comparator, and an LDO, any of a variety of schematics and circuit architectures may be utilized as appropriate to the requirements of specific applications in accordance with embodiments of the invention.

The power consumption of the each block, including the voltage reference, voltage divider and comparator illustrated in FIGS. 13a, 13b, and 13c is shown in Table I below. In certain embodiments, the total power consumption in sleep mode is less than 200 nA, when V_{DD} is 1 V.

TABLE I
Current Consumption

| | Current Consumption |
|---|---|
| | in Sleep Mode |
| Voltage Reference | 43 nA |
| Voltage Divider | 5 nA |
| Comparator | 110 nA |

### Dielectric Sensing Sensor

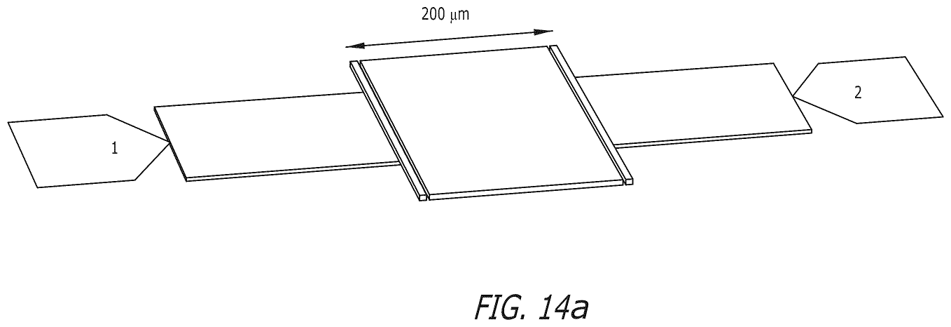

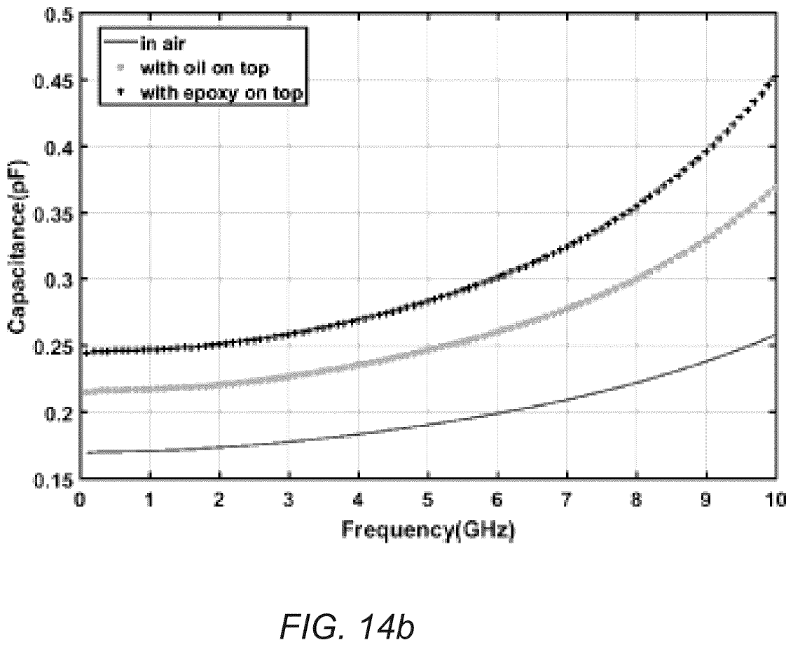

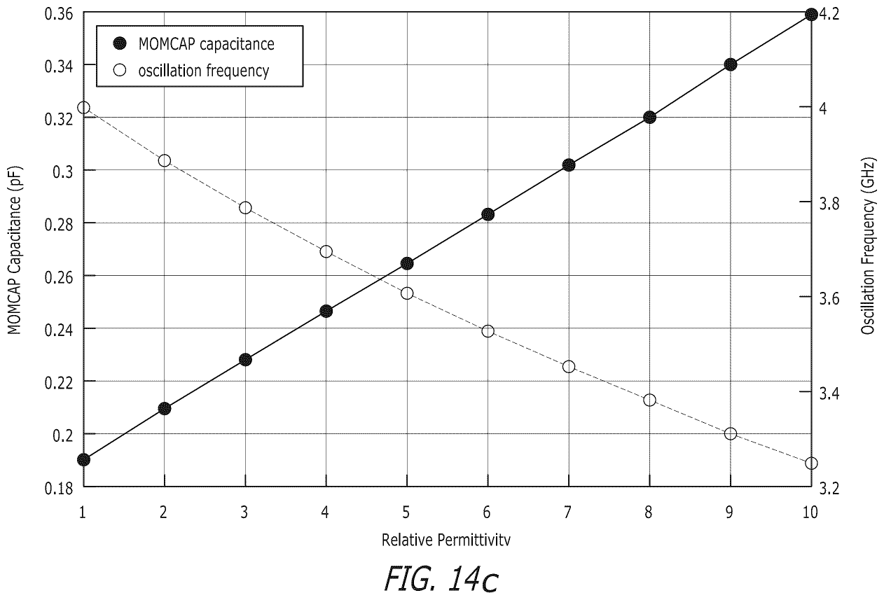

In many embodiments, the dielectric sensing can be performed by a customized metaloxide-metal capacitor (MOMCAP) which may be implemented using the top metal layer in this process. FIG. 14a illustrates a MOMCAP structure in 180 nm CMOS top layer in accordance with an embodiment of the invention. The MOMCAP may present different capacitance with the different target material on its top. A LC cross-coupled pair oscillator can be designed utilizing the MOMCAP in resonant tank, which translates the capacitance into oscillation frequency. The oscillator may drive another on-chip dipole antenna to transmit this signal back to external receiver.

In many embodiments, the MOMCAP size can be 200 by 200 μm, including 10 fingers on each side, with a finger width and gap distance of 5 μm. The silicon substrate thickness may be 250 μm, with a 14 μm silicon dioxide on top. The passivation layer above the MOMCAP may be removed to improve the sensitivity to the change in the dielectric constant of the material placed on the chip. The MOMCAP capacitance can be simulated in EM simulator Hyperlynx 3DEM is about 184 fF when the chip is placed in air as illustrated in FIG. 14a in accordance with several embodiments of the invention. The effective capacitance may increase if a high dielectric constant material is placed top of MOMCAP, causing a lower oscillation frequency. In certain embodiments, the oscillation frequency may be set at 4 GHz when the dielectric sensor chip is placed in the air. The oscillator may consume 4 mW during oscillation. The capacitance of MOMCAP with olive oil (dielectric constant of 3.1) and epoxy (dielectric constant of 5) on top are also simulated and reported in FIG. 14b in accordance with several embodiments of the invention. FIG. 14c illustrates that as dielectric constant increases, the MOMCAP capacitance may increase leading to a smaller oscillation frequency in accordance with several embodiments of the invention. In certain embodiments, the oscillator may directly drive another on-chip dipole antenna at around 4 GHz. The antenna radiation efficiency can be 4% at 4 GHz. Although FIG. 14a illustrates a particular MOMCAP structure and size fabricated in 180 nm CMOS top metal layer, any of a variety of structures, sizes, and fabrication processes may be utilized as appropriate to the requirements of specific applications in accordance with embodiments of the invention.

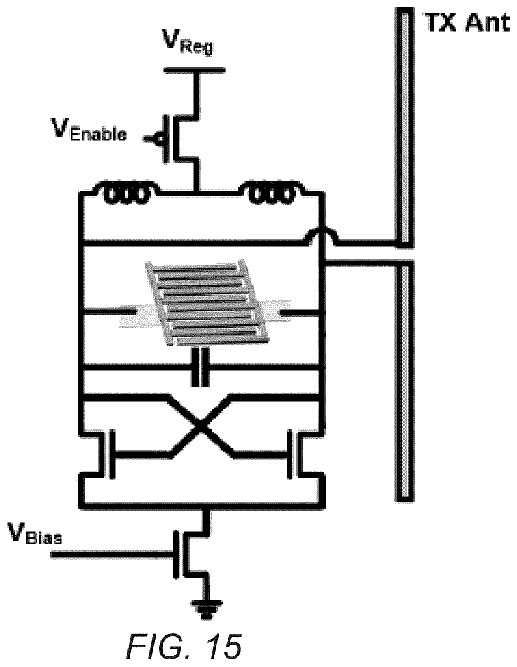

In certain embodiments, at this supply level, the oscillator may generate −25 dBm power to the TX on-chip antenna. In simulation, the free-running frequency can be 4 GHz. A schematic of an LC dielectric sensing sensor in accordance with several embodiments of the invention is illustrated in FIG. 15. Although FIG. 15 illustrates a particular schematic of an LC dielectric sensing sensor, any of a variety of architectures may be utilized as appropriate to the requirements of specific applications in accordance with embodiments of the invention. Additional details for designing dielectric sensing sensors, circuit design and techniques as related to, for example, FIG. 15, can be found in Pozar, David M. “Microwave engineering” John Wiley & Sons, 2009, Chapter 13, “Oscillators and Mixers”, starting on page 604, and in Razavi, Behzad “RF microelectronics” Vol. 1, New Jersey: Prentice hall, 1998, Chapter 8 starting on page 497, and in Cheng, David Keun, “Field and wave electromagnetics”, Pearson Education India, 1989, and in Razavi, Behzad, “Design of analog CMOS Integrated Circuits” 2001, the disclosures of which are hereby incorporated by reference in their entirety.

### Measurement Result

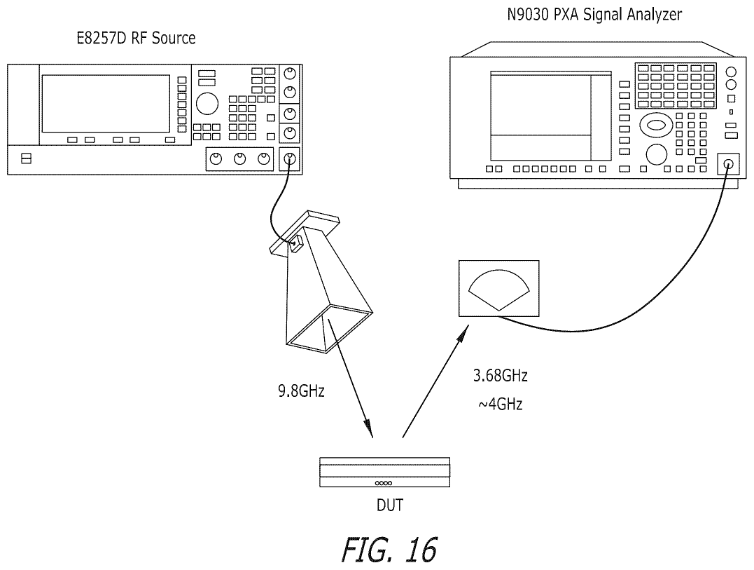

Described below are certain measurement results for a dielectric sensing circuit in accordance with several embodiments of the invention. FIG. 16 illustrates a measurement setup in accordance with several embodiments of the invention. A Keysight E8257D RF source may generate an RF signal that is amplified by an X-band PA and then fed to a horn antenna. The horn antenna can be used to transmit electromagnetic waves with EIRP of 36 dBm at 9.8 GHz. The chip may be placed 6 cm away. The microchip may harvest the power and transmit the oscillator freerunning signal back to the RX slot-bowtie antenna in the frequency range of 3.6 GHz to 4 GHz. The receiving antenna can be connected to a Keysight PXA N9030A spectrum analyzer.

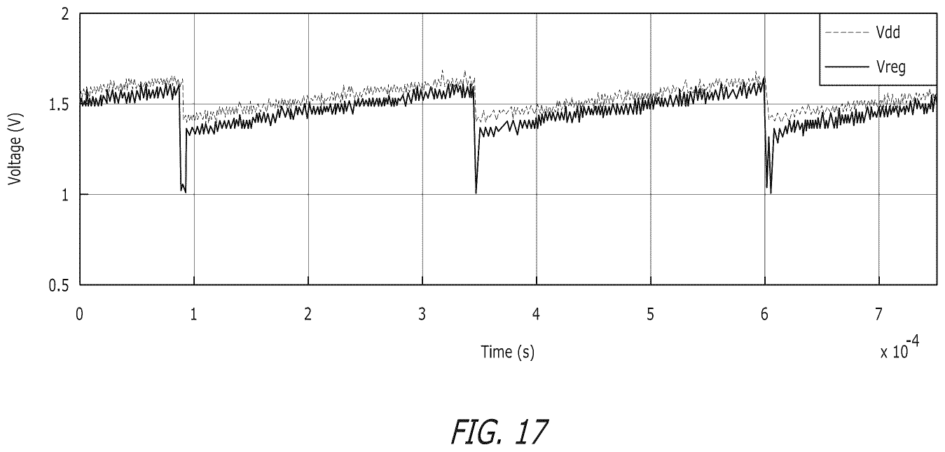

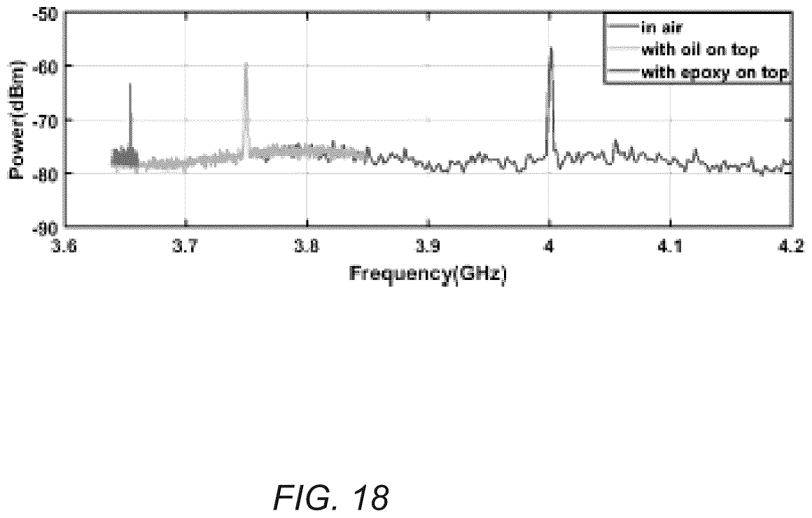

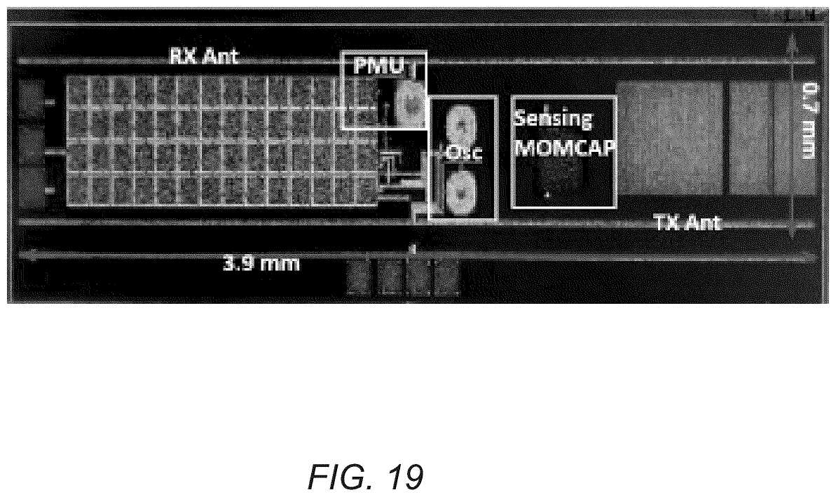

Transient signal of the voltage on storage capacitor and regulated voltage at LDO output in accordance with several embodiments of the invention are plotted in FIG. 17. As illustrated in FIG. 17, when the Vad voltage is charged to 1.65 V, it may turn on the oscillator. Then the storage capacitor can be discharged to 1.3 V. The repetition rate can be tuned by adding an external storage capacitor or tuning the received RF power. The LDO regulated voltage may be 1 V, which can be used as the supply voltage for the oscillator. The received signal spectrum in accordance with several embodiments of the invention is illustrated in FIG. 18, demonstrating the oscillation frequency of the chip in different environments. The free-running frequency in air can be 4 GHZ. The frequency may change to 3.75 GHz when oil is placed on top of the chip. It may change 3.66 GHZ when an epoxy is used. This indicates the MOMCAP capacitance may increase to 236 fF and 265 fF when oil or epoxy is used. These values match well with the EM simulation results. A die micrograph of a dielectric sensor chip in accordance with several embodiments of the invention is illustrated in FIG. 19. The total chip size can be only 2.73 mm² that includes integrated on-chip dipole antennas. In many embodiments, the chip can be fabricated in a 180 nm CMOS SOI process and occupies an area of 3:9 0:7 mm², including the on-chip antennas and storage capacitor. The die photo of the chip in accordance with several embodiments is illustrated in FIG. 19. Although FIG. 19 illustrates a particular dielectric sensing chip fabricated in a 180 nm CMOS SOI process, any of a variety of chip sizes and fabrication processes may be utilized as appropriate to the requirements of specific applications in accordance with embodiments of the invention.

Accordingly, many embodiments provide a fully integrated wirelessly powered frequency-shift based dielectric sensor with on-chip antennas. In many embodiments, the sensor includes an energy-harvesting frontend, a power management unit, a dielectric sensing sensor, and on-chip receiving and transmitting antennas. In many embodiments, the chip has the ability to sense different dielectric material such as oil and epoxy by measuring the frequency shift of the oscillator. In many embodiments, the entire chip may be powered wirelessly through an on-chip antenna. In certain embodiments, the chip may have a maximum operating distance of 6 cm.

Although specific methods and systems for battery-less wirelessly powered dielectric sensors are discussed above, many different systems can be implemented in accordance with many different embodiments of the invention. It is therefore to be understood that the present invention may be practiced in ways other than specifically described, without departing from the scope and spirit of the present invention. Thus, embodiments of the present invention should be considered in all respects as illustrative and not restrictive. Accordingly, the scope of the invention should be determined not by the embodiments illustrated, but by the appended claims and their equivalents.

What is claimed is:

1. A wirelessly powered dielectric sensor, comprising:
an RF-power receiving antenna that receives electromagnetic power;
a power management unit (PMU) comprising a capacitor to rectify and store the electromagnetic power; and
a dielectric constant sensing sensor;
wherein the PMU monitors harvested energy and operates the dielectric sensing sensor;
wherein the dielectric sensing sensor senses a dielectric constant of a material that is in close proximity.
